(19) United States
(12) Patent Application Publication
(10) Pub. No.: US 2010/0063530 A1
(43) Pub. Date: Mar. 11, 2010

(54) INTRA-GASTRIC BALLOON WITH DOUBLE-MEMBRANE VALVE AND CORRESPONDING KIT FOR SETTING SAME

(76) Inventor: Nicolas Francois Michel Valencon, Pont Eveque (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/916,164

(22) PCT Filed: Jun. 1, 2005
(86) PCT No.: PCT/FR2005/001343

§ 371 Date: Aug. 26, 2008

(51) Int. Cl. A61M 29/02

(52) U.S. Cl. 606/192

(57) ABSTRACT

The invention relates to an intra-gastric balloon comprising:
at least one bag suitable for being filled, at least partially. With an inflation fluid; and
connection means (3) including a self-closing member (21) suitable fear being transpierced by a connection member for connection to a inflation fluid source;
said intra-gastric balloon being characterized in that the self-closing member (21) comprises at least two superposed self-healing membranes (23), forming one or more pairs of self-healing membranes (23) suitable for being transpierced in succession by the connection member, each self-healing membrane (23) being mounted in a manner Such as to come to bear against the other self-healing membrane (23) of the pair with a view to forming a leaktight barrier suitable for automatically closing the passage formed through said other self-healing membrane (23) of the pair, once the connection member has been removed.

The invention also provides an intra-gastric balloon for treating obesity.

### TECHNICAL FIELD

The present invention relates to the general technical field of devices that are implantable in the human body and that are designed to be used in treating obesity, in particular morbid obesity, and the present invention relates especially to implants that are suitable for artificially reducing the volume of the stomach, in particular with a view to procuring a feeling of satiety in the patient.

The present invention relates more particularly to an intra-gastric balloon designed to be implanted inside the stomach of a patient for the purpose of reducing the volume of the stomach by way of treatment for obesity, said balloon comprising:

at least one bag suitable for being filled, at least partially, with an inflation fluid; and

connection means, mounted on said bag and including a self-closing member suitable for being transpierced by a connection member for connection to a inflation fluid source.

The present invention also relates to a kit for deploying an intra-gastric balloon designed to be implanted in the stomach of a patient by way of treatment for obesity, said kit comprising:

an intra-gastric balloon; and

a connection member for connecting the intra-gastric balloon to an inflation fluid source.

### PRIOR ART

In order to treat patients suffering from obesity, it is known that intra-gastric balloons can be used that are designed to be implanted in the stomachs of the patients in order to reduce the space available for food, with a view, in particular to procuring a feeling of satiety.

Such a known intra-gastric balloon generally comprises at least one flexible bag suitable for taking up a collapsed (or small-volume) position enabling the intra-gastric balloon to be implanted via the oral route.

The bag is designed to be filled, once the intra-gastric balloon is implanted in the stomach, with an inflation fluid, e.g. with air or with physiological fluid, in a manner such as to impart to the intra-gastric balloon its operational shape, in which it occupies a volume inside the stomach that is sufficient to occupy a large portion of the space available for food.

The bag is generally made of materials having good impermeability properties so as to prevent the fluid contained in the bag from progressively leaking into the stomach and so as to prevent the intra-gastric balloon from deflating. In particular, the leaktightness of the bag must be sufficient for the intra-gastric balloon to maintain its operational shape throughout the period of the treatment that can vary from a few weeks to several months.

In known intra-gastric balloons, the bag is provided with connection means, such as a valve, designed to enable the intra-gastric balloon to be connected to an inflation fluid source, via a connection member comprising, for example, a catheter and an inflation needle. The connection means further comprise means for guaranteeing that it is leaktight, once the connection member has been withdrawn, in order to prevent the fluid contained in the bag from leaking out.

When the inflation fluid is formed by a liquid, such as a physiological fluid, the means used can be in the form of a “duck-bill” valve. A duck-bill valve is designed in such a manner as to allow pressurized fluid to flow from the outside towards the inside of the intra-gastric balloon, and to prevent fluid from flowing in the opposite direction. In general, such valves make it possible to obtain good results with liquids, but they are generally unsatisfactory when the inflation fluid is formed by a gas. The leaktightness procured with that type of valve is then no longer satisfactory. Valves of the “septum” type are then preferred. Such a septum valve is formed by a membrane that is made of silicone, that can be of various thicknesses, that is suitable for being transpierced by an inflation needle, and that has self-healing characteristics.

Intra-gastric balloons equipped with valves of the “septum” type are simple to manufacture and are generally satisfactory, but they do however suffer from various non-negligible drawbacks.

Firstly, in the event of prolonged storage, an aging phenomenon can appear in the silicone forming the septum. In particular, the septum can become dry and brittle, and lose its elasticity and its impermeability properties under the effect of such aging.

In addition, intra-gastric balloons are sometimes sold in kits, such a kit including an inflation needle pre-installed on the balloon, the inflation needle being pre-positioned, in its operational position, so as to pass through the septum and so as to open out into the bag of the intra-gastric balloon.

Such kits can be stored in sterile or non-sterile housings for prolonged periods before being used. Unfortunately, the prolonged presence of the inflation needle through the septum can give rise to formation of an orifice which, if the storage period is very long, can remain after the needle has been removed, once the intra-gastric balloon has been inflated. Such an orifice then constitutes a preferred passage via which fluid contained in the intra-gastric balloon can leak out, thereby causing the balloon to deflate.

### SUMMARY OF THE INVENTION

Objects assigned to the invention are thus to remedy the various above-listed drawbacks, and to propose a novel intra-gastric balloon of leaktightness that is greater than the leaktightness of prior art intra-gastric balloons, even after a prolonged period of storage.

Another object of the invention is to propose a novel intra-gastric balloon that is suitable for being connected easily, in leaktight manner, and entirely safely to an inflation fluid source.

Another object of the invention is to propose a novel intra-gastric balloon that is particularly lightweight and well tolerated by the patient.

Another object of the invention is to propose a novel intra-gastric balloon that can be extracted easily and rapidly via the oral route.

Another object of the invention is to propose a novel intra-gastric balloon that is suitable for being grasped easily and securely by endoscopic extractor tools.

Objects assigned to the invention are also to propose a novel kit comprising an intra-gastric balloon and a connection member for connecting the balloon to an inflation fluid source, and that, even after a long period of storage, offers good leaktightness properties.

The objects assigned to the invention are achieved by means of an intra-gastric balloon designed to be implanted inside the stomach of a patient for the purpose of reducing the volume of the stomach by way of treatment for obesity, said balloon comprising:

at least one bag suitable for being filled, at least partially, with an inflation fluid; and

connection means, mounted on said bag and including a self-closing member suitable for being transpierced by a connection member for connection to a inflation fluid source;

said intra-gastric balloon being characterized in that the self-closing member comprises at least two superposed self-healing membranes, forming one or more pairs of self-healing membranes suitable for being transpierced in succession by the connection member, each self-healing membrane being mounted in a manner such as to come to bear against the other self-healing membrane of the pair with a view to forming a leaktight barrier suitable for automatically closing the passage formed through said other self-healing membrane of the pair, once the connection member has been removed.

The objects assigned to the invention are also achieved by means of a kit for deploying an intra-gastric balloon designed to be implanted in the stomach of a patient by way of treatment for obesity, said kit comprising:

an intra-gastric balloon of the invention, and at least as described above; and

a connection member for connecting the intra-gastric balloon to the inflation fluid source, which connection member is, in its storage configuration, mounted in a manner such as to pass through the superposed self-healing membranes so as to open out into the bag.

### BRIEF DESCRIPTION OF THE DRAWINGS

Other objects and advantages of the invention will appear on reading the following description, and on examining the accompanying drawings which are given merely by way of non-limiting illustration and in which:

FIG. 1 is a section view showing an intra-gastric balloon of the invention that has a single bag, with connection means provided with a self-closing member that is being transpierced by an inflation needle;

FIG. 2 is a section view showing a variant embodiment of an intra-gastric balloon of the invention that has two concentric bags;

FIG. 3 is a perspective view showing connection means mounted on an intra-gastric balloon of the invention;

FIG. 4 is a section view on line A-A of FIG. 3, showing the connection means shown in FIG. 3;

FIG. 5 is a diagrammatic section view showing a detail of the self-closing member shown in FIG. 1;

FIG. 6 is a diagrammatic section view showing the detail of FIG. 5 after the inflation needle has been removed; and

FIG. 7 is a section view showing a variant embodiment of the connection means of the invention.

### BEST MANNER OP IMPLEMENTING THE INVENTION

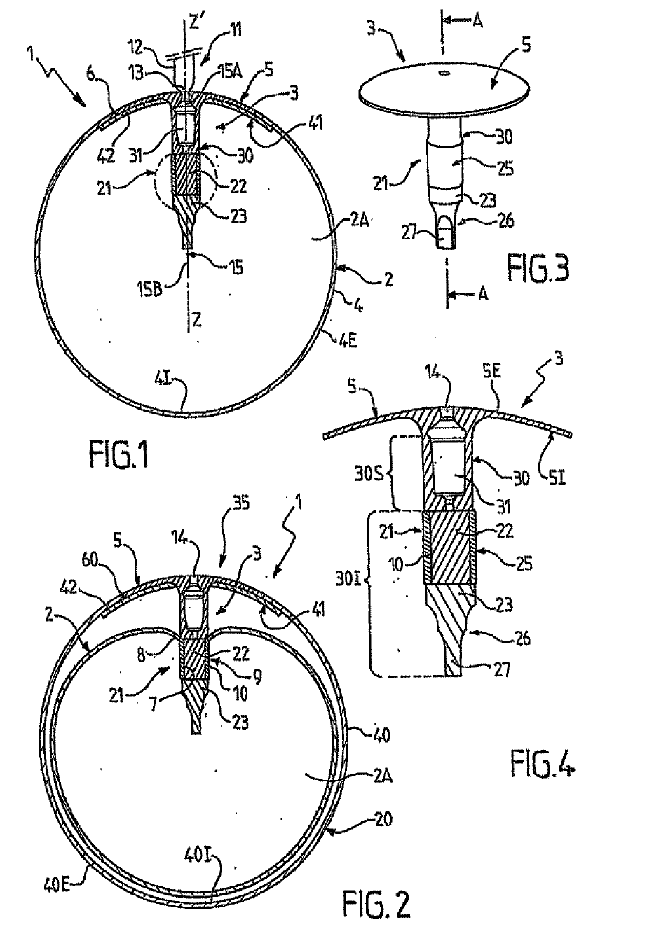

FIGS. 1 and 2 show two variant embodiments of an intra-gastric balloon 1 of the invention in its operational configuration, i.e. in its expanded configuration, in which it occupies a large portion of the space available for food in the stomach.

The intra-gastric balloon 1 of the invention advantageously has the characteristic of being expandable, i.e. it is made of flexible materials, and preferably of elastomer materials such as silicone or elastomer thermoplastic polyurethane, enabling it to expand from a collapsed configuration (not shown), in which it occupies a small volume enabling it to be implanted via the oral route, to an expanded configuration in which it occupies an operational volume (FIGS. 1 and 2).

The intra-gastric balloon 1 of the invention comprises at least one bag 2 suitable for being filled, at least partially, with an inflation fluid, and defining an internal volume 2A.

The intra-gastric balloon 1 of the invention further comprises at least one set of connection means 3 mounted on said bag 2 and designed to be connected to a fluid source (not shown) by means of a connection member 11 in order to cause the intra-gastric balloon 1 to expand inside the stomach by being filled with the inflation fluid.

In a first embodiment of the invention shown in FIG. 1, the intra-gastric balloon 1 comprises a single bag 2 forming a surface casing 4 for the intra-gastric balloon 1. In preferred manner, the bag 2 is made of a bio-compatible flexible material, and preferably of medical-grade silicone.

In this first embodiment, the connection means 3 are mounted on the bag 2 and are preferably secured thereto, e.g. by bonding with adhesive or by heat-sealing. To, this end, the connection means 3 are provided with a collar 5 designed to enable the connection means 3 to be fastened to the bag 2 in leaktight manner, e.g. by bonding the collar 5 with adhesive or by heat-sealing it to the periphery of a hole 6 provided through the bag 2.

In a second embodiment shown in FIG. 2, the intra-gastric balloon 1 of the invention comprises at least first and second bags 2, 20 that are preferably flexible, the first bag 2 being disposed inside the second bag 20 so as to form an inner first bag 2 and an outer second bag 20. In this variant, the connection means 3 are fastened in leaktight manner to the first bag 2, and are more precisely mounted in a passage 7 defined by a neck 8 extending towards the inside of the first bag 2. More precisely, the connection means 3 are fastened to the first bag 2 by means of a fastening element 9 that is preferably formed by a ring 10. The fastening element 9 is thus advantageously shaped and disposed so as to exert pressure on the neck 8 that is sufficient to pinch it between the connection element 3 and the fastening element 9.

In the second embodiment of the invention, the first bag 2 forms means for deploying the second bag 20, said second bag then forming the surface casing 40 of the intra-gastric balloon 1. The inflation fluid, and in particular air, is advantageously fed into the first bag 2, thereby causing it to inflate. In the manner of a tire inner tube, the first bag 2 expanding in turn causes the second bag 20 forming the surface casing 40 of the intra-gastric balloon 1 to expand and to be deployed.

Advantageously, the first and second bags 2, 20 are made of materials that are mutually different and that are not necessarily compatible.

The expression “non-compatible” refers to materials that are particularly difficult or even impossible to assemble together conventionally by adhesive bonding or by heat-sealing, in view of industrial production constraints and of medical requirements as regards quality and safety.

Preferably, the first and second bags 2, 20 are made of elastomer materials, the first bag 2 preferably being made of a material having a gas barrier effect, such as elastomer thermoplastic polyurethane, and the second bag preferably being made of a biocompatible material having good strength, such as silicone. The use of a barrier-effect material for the first bag 2 advantageously makes it possible to reduce the thickness of the first bag 2 while preserving or even improving the leaktightness of the balloon.

In the second embodiment of the invention, the periphery of the collar 5 is preferably bonded with adhesive or heat-sealed to the hole 60 provided through the second bag 20, the connection means 3 thus closing off in leaktight manner the second bag 20, i.e. the surface casing 40 of the intra-gastric balloon 1. The two-bag configuration shown in FIG. 2 is preferred to the one-bag configuration shown in FIG. 1 because it makes it possible to obtain an intra-gastric balloon 1 that has strength and leaktightness that are higher than those of single-bag balloons, while remaining compact in the collapsed state, in particular by means of the small thickness of the inner bag.

The connection member 11 advantageously comprises a catheter 12 serving to provide fluid connection between the intra-gastric balloon 1 and the inflation fluid source and designed to be connected to the intra-gastric balloon 1 by means of an end-piece 13 inserted into a recess 14 provided in the connection means 3. The connection member 11 further comprises an inflation needle 15 that is preferably formed by a hollow needle having one of its ends 15A secured to the end-piece 13 and its other end 15B, which is preferably atraumatic, situated inside the internal volume 2A. As shown in FIG. 1, the inflation needle is inserted through the connection means 3 along a perforation axis Z-Z′.

In accordance with the invention, the connection means 3 include a self-closing member 21 suitable for being transpierced by the connection member 11 and more precisely by the inflation needle 15 (FIG. 1). Advantageously, the connection means 3 include a main body 30 that is preferably elongate and that extends towards the inside of the intra-gastric balloon 1. The self-closing member 21 is then preferably formed on the main body 30 and is thus situated inside the intra-gastric balloon 1, in the internal volume 2A.

In accordance with the invention, the self-closing member 21 comprises at least two self-healing membranes 22, 23 that are mutually superposed and that form one or more pairs of self-healing membranes 22, 23. In accordance to the invention, the self-healing membranes 22, 23 are designed and disposed in a manner such as to be transpierced in succession by the connection member 11 and in particular by the inflation needle 15. By passing through the self-healing membranes 22, 23, the inflation needle 15 thus forms a passage 24 through each membrane.

The expression “self-healing membrane” refers to a membrane (or wall) made of a material that is sufficiently flexible to be transpierced by the connection member 11, and in particular by the inflation needle 15, and that has elasticity and shape memory properties that are sufficient to close the passage 24 left after the inflation needle 15 has been removed, thereby forming a leaktight membrane.

In accordance with an essential characteristic of the invention, each self-healing membrane 22, 23 is mounted in a manner such as to bear against the other self-healing membrane 22, 23 of the pair with a view to forming a leaktight barrier suitable for automatically closing the passage 24 formed through said other self-healing membrane 22, 23 of the pair once the connection member has been removed. The association of the two self-healing membranes 22, 23 of the invention thus imparts to the self-closing member 21 its self-closing properties, i.e. its capacity to close automatically and without any external action once the connection member 11 and more precisely the inflation needle 15 has been removed.

The self-closing properties of the self-closing member 21 are obtained by the combination of two distinct effects, namely:

the self-healing characteristic specific to each self-healing membrane 22, 23 that makes it possible for said self-healing membranes to self-heal and to close independently of each other by closing the passage 24 formed after the connection member 11 has been removed; and

the mutual closure action exerted by each self-healing membranes 22, 23 on the other self-healing membrane 22, 23 of the pair.

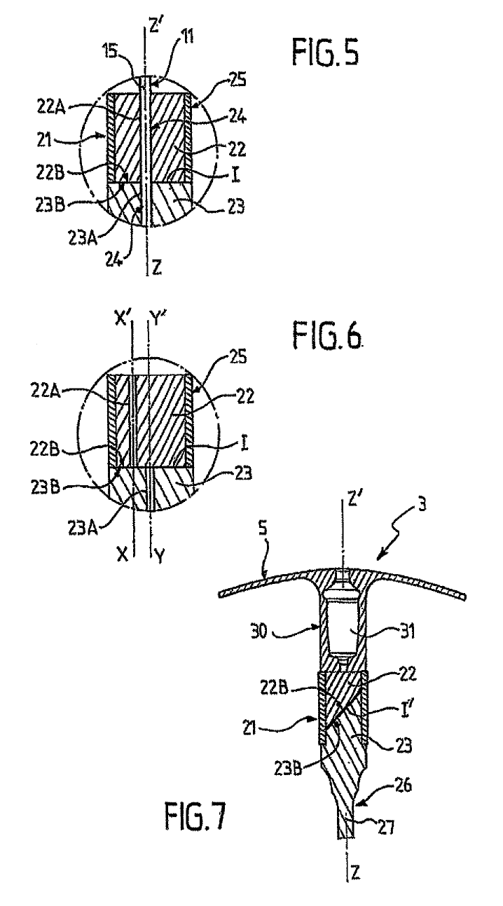

As shown in FIG. 5, the passage 24 formed through the self-healing member 21 by the connection member 11 and more precisely by the inflation needle 15 is made up of two sub-passages 22A, 23A formed in respective ones of first and second self-healing membranes 22, 23 forming the above-mentioned pair of membranes.

The resulting two sub-passages 22A, 23A, which are initially aligned with the perforation axis Z-Z′ of the inflation needle 15, do not remain in axial alignment with each other once the inflation needle 15 has been removed. In other words, under the effect of the self-healing membranes 22, 23 returning elastically to their initial configuration, as allowed by the inflation needle being removed, the respective axes X-X′ and Y-Y′ along which the sub-passages 22A, 23A extend, are offset laterally as shown in FIG. 6. By means of this offset, the sub-passage 22A formed through the first self-healing membrane 22 is then closed by the second self-healing membrane 23 while the sub-passage 23A formed through the second self-healing membrane 23 is closed by the first self-healing membrane 22, each self-healing membrane 22, 23 then forming a leaktight barrier against the facing sub-passage 22A, 23A.

Particularly advantageously, the self-healing membranes 22, 23 thus come to bear in leaktight manner against each other in order to close the sub-passages 22A, 23A mutually. The self-healing membranes 22, 23 are not necessarily compressed against each other, it being possible for mere passive abutment to suffice to guarantee leaktightness at the interface I between the self-healing membranes 22, 23.

Naturally, it is also possible, without going beyond the ambit of the invention, to imagine the self-closing member 21 having more than two superposed self-healing membranes, e.g. three or four self-healing membranes.

The association of two distinct self-healing membranes 22, 23 in order to form the self-closing member 21 is particularly advantageous when the connection member 11 and in particular the inflation needle 15 is mounted through the self-closing member 21 for a prolonged period, e.g. in the event of long-term storage. In which case, it is possible to observe a reduction in the self-healing capability that is specific to each self-healing membrane 22, 23. This phenomenon can, in particular result from aging of the material (e.g. silicone) of which the membranes are made, which material becomes dry and brittle and loses its elasticity properties. The passage 24, and in particular the sub-passages 22A, 23A then might remain open and might not heal once the inflation needle 15 has been removed.

In prior art intra-gastric balloons, in which the self-closing member 21 is formed by a single membrane, the non-healed passage therethrough then constitutes a non-negligible source of leakage of the inflation fluid contained in the internal volume 2A. Conversely, with the structure of the self-healing member 21 of the invention, the self-healing member 21 retains its self-closing properties even if the self-healing membranes 22, 23, due to a reduction in their intrinsic self-healing capability, are no longer capable individually of guaranteeing their own healing.

Certain parameters can contribute to increasing the statistical probability of obtaining an offset between the sub-passages 22A, 23A after the inflation needle has been removed, and to increasing the amplitude of said offset.

In particular, the difference in hardness or elasticity between the self-healing membranes 22, 23 constitutes a first parameter that can facilitate the offsetting of the sub-passages 22A, 23A. If the self-healing membranes 22, 23 do not have the same elasticity and hardness properties, they behave differently when the inflation needle 15 is removed, thereby contributing to the mutual offset between the sub-passages 22A, 23A.

In general, a different rest state for each self-healing membrane 22, 23 can contribute to offsetting the sub-passages 22A, 23A once the inflation needle 15 has been removed. In particular, each self-healing membrane 22, 23 can, in the rest configuration, i.e. before it is transpierced by the inflation needle 15, find itself in a relaxed state or in a compressed state. Depending on whether it is in a relaxed state or in a compressed state, each self-healing membrane 22, 23 does not have the same behavior once the inflation needle has been removed. This difference in behavior then increases the statistical probability of an offset being observed between the sub-passages 22A, 23A.

Advantageously, the self-healing membranes 22, 23 are formed of materials that have shape memory and that have substantially the same flexibility and elasticity properties in a manner such as to guarantee mutual leaktightness for them. Preferably, the self-healing membranes 22, 23 are made of an elastomer material such as silicone. Even more preferably, the self-healing membranes are made of the same material (preferably silicone) in order to optimize the leaktightness at the interface I. At the interface I, silicone-on-silicone contact is thus obtained that offers a leaktight characteristic.

Naturally, it is possible to imagine using any type of material, other than silicone, that has flexibility, impermeability, and elasticity properties that are sufficient for being transpierced by a needle and for self-healing, once the needle has been removed.

In a variant embodiment of the invention, the self-healing membranes 22, 23 are made of materials (e.g. based on silicones) of different hardnesses.

In a first embodiment of the invention, the self-healing membranes 22, 23 are mounted to be free, at least locally, relative to each other, so as to make it possible, at least locally, for them to move along each other. Preferably, the self-healing membranes 22, 23 are mounted to be free relative to each other at least in a perforation zone in which they are perforated by the inflation needle 15. Such a mounting configuration thus facilitates offsetting of the sub-passages 22A, 23A once the inflation needle has been removed. Such a configuration is not however necessary in order to obtain the offset.

In a second preferred embodiment of the invention, the self-healing membranes 22, 23 are attached to each other. The mutual attachment of the self-healing membranes 22, 23 is not sufficient to prevent the sub-passages 22A, 23A from being offset to a small extent. Thus, it is observed that the sub-passages 22A, 23A do not coincide after the connection member 11 has been removed even though the self-healing membranes 22, 23 are attached to each other.

The self-healing membranes 22, 23 advantageously come to bear against each other, at the interface I, via at least two contact faces 22B, 23B, said contact faces 228, 235 being fastened together, e.g. by adhesive bonding or by heat-sealing even more preferably, the contact faces 225, 235 are fastened together over substantially their entire area.

Another embodiment of the self-closing member 21, shown in FIG. 7, contributes to increasing the statistical probability of obtaining an offset between the sub-passages 22A, 23A. In this embodiment, the two self-healing membranes 22, 23 came to bear against each other at an interface I′ that is inclined relative to the perforation axis Z-Z′, i.e. at an interface that is not perpendicular to the perforation axis Z-Z′. The contact faces 22B, 235 are then inclined relative to the perforation axis Z-Z′, and they form an acute angle therewith.

Advantageously, and as shown in the figures, the self-closing member 21 further comprises a compression member 25, of the ring type, disposed in a manner such as to surround at least one of the self-healing membranes 22, 23, in a manner such as to compress it, preferably laterally and inwardly. The compression member 25 and the fastening member 9 are preferably formed by the same element, namely the ring 10.

Preferably, the pair of self-healing membranes 22, 23, is formed by a first self-healing membrane 22 that is compressed by the compression member 25 and by a second self-healing membrane 23 that is not compressed by the compression member 25, and thus that is at rest, i.e. in a relaxed state (FIG. 4). The first and second self-healing membranes 22, 23 then behave differently from each other after the connection member 11 has been removed, thereby facilitating the offsetting of the sub-passages 22A, 23A.

In addition, the use of a compression member 25 also makes it possible to improve the specific self-healing capability of the first self-healing membrane 22.

When the intra-gastric balloon 1 is in the “vertical” position, as shown in FIGS. 1 and 2, the connection means 3 and the main body 30 extend in a substantially vertical direction that coincides with the perforation axis Z-Z′. In this vertical configuration, the first self-healing membrane 22, which is compressed, is preferably situated above the second self-healing membrane 23, which is relaxed.

Advantageously, the main body 30 comprises a top portion 30S that is hollow so as to form a cavity 31, e.g. a cylindrical cavity, and a bottom portion 30I including the self-healing member 21.

It is interesting to note that the presence of the cavity 31 in the connection means 3, and in particular in the main body 30, is independent of whether or not the two self-healing membranes 22, 23 are present. The purpose of the cavity 31 is merely to facilitate taking hold of the intra-gastric balloon 1 when extracting it and said cavity could thus be associated with any type of self-closing member and, for example, with a single self-healing membrane.

In order to facilitate taking hold of the intra-gastric balloon 1, the top portion 30S is thus hollow over a distance sufficient for the wall (or the walls) of the main body 30 defining the cavity 31 to be able to fold over while the top portion 30S of the main body 30 is being pinched with an extractor tool (not shown) for extracting the intra-gastric balloon 1, such as endoscopic forceps.

Preferably, the cavity 31 extends longitudinally, along the perforation axis Z-Z′, over a length greater than its mean width (or than its diameter, when the cavity is a cylindrical cavity) measured in a direction that is substantially perpendicular to the perforation axis Z-Z′.

By way of illustrative and non-limiting example, the cavity 31 can be of length greater than 1.5 times its mean width.

The length of the cavity 31 advantageously results from a compromise between:

firstly the need to limit the total length of the connection means 3 and of the main body 30 so as not to hinder implantation of the intra-gastric balloon 1 via the oral route; and

secondly, the need to be able to pinch the connection means 3 in the vicinity of the surface casing 4, 40 of the intra-gastric balloon 1 in order to enable said balloon to be extracted.

Thus, the intra-gastric balloon 1 is implanted while the inflation needle 15 is mounted in the connection means 3 in a manner such as to pass longitudinally through the main body 30. The main body 30 and the inflation needle 15 thus form a rigid assembly having very low longitudinal flexibility. However, in order to insert the intra-gastric balloon 1 into the oral passageways of the patient, the surgeon requires a certain amount of flexibility, hence the need to minimize the length of the main body 30.

Preferably, the collar 5 extends radially to the surface of the intra-gastric balloon 1 in a manner such as to form a graspable zone 35 via which the intra-gastric balloon 1 can be taken hold of by the extractor tool (not shown).

It is possible to imagine making the collar 5 of a material having hardness greater than the hardness of the material forming the surface casing 4, 40, e.g. of hard silicone, so as to offer, at least locally, higher strength, and so as to prevent it from being transpierced by the extractor tool. However, it is preferable, in order to enable the intra-gastric balloon 1 to collapse, to make the collar 5 of a flexible material of hardness equivalent to or only very slightly greater than the hardness of the surface casing 4, 40.

As shown in FIGS. 1 and 2, the graspable zone 35 of the intra-gastric balloon 1 is preferably associated with a reinforcement 41 for forming a reinforced graspable zone 35 dedicated to extracting the intra-gastric balloon 1. The reinforcement 41 is advantageously in the form of a flexible membrane 42 that extends along the graspable zone 35 in a manner such that it does not project towards the outside of the surface casing 4, 40, and thus does not form any protuberance that might be detrimental to the uniform and atraumatic geometrical shape of the intra-gastric balloon 1.

Preferably, the shape of the reinforcement 41 substantially matches the shape of the collar 5 and the shape of the surface casing 4, 40, the reinforcement being associated with said collar and with said casing.

Even more preferably, the reinforcement 41 is superposed on the collar 5 inside the intra-gastric balloon 1 and is advantageously bonded with adhesive or heat-sealed over its entire surface to the inside surface 51 of the collar 5. The reinforcement 41 can also be extended, as shown in FIGS. 1 and 2, under the surface casing 4, 40. In which case, the reinforcement 41 is preferably secured to the surface casing 4, 40 by being heat-sealed or bonded with adhesive to the inside face 4I, 40I of said surface casing 4, 40. It is also possible, without going beyond the ambit of the invention, to imagine mounting the reinforcement 41 on the outside surface 5E of the collar or indeed embedding the reinforcement 41 in the thickness of the collar 5. Similarly, the reinforcement 41 may extend over the outside face 4E, 40E of the surface casing 4, 40 or be embedded in the thickness thereof.

The reinforcement 41 preferably comprises at least one piece of textile, comprising, for example, a polyester net, or indeed a woven (or non-woven) fabric made from polyamide fibers and/or aramid fibers. It is also possible to imagine implementing, as a reinforcement piece, a fibrous structure of the “honeycomb” type that is well known per se.

Advantageously, the self-closing member 21 includes a pull member 26 suitable for being manipulated for axially pulling the self-closing member 21, in particular in a main extension direction in which it extends parallel to the perforation axis Z-Z′, and for enabling the compression member 25 to be mounted around at least one of the self-healing membranes 22, 23. Thus, the compression member 25 advantageously has an inside diameter smaller than the outside diameter at rest of the self-healing membrane 22 with which it is associated so that it can radially compress said membrane inwardly.

Axially pulling the self-healing membrane 22 with which the compression member 25 is associated then makes it possible to reduce the outside diameter of the self-healing membrane 22 until said outside diameter reaches the inside diameter of the compression member 25 and in particular of the ring 10. It is then possible to fit the ring 10 over the self-healing membrane 22, as shown in FIG. 4.

Preferably, the pull element 36 is formed by a traction tab 27 that is secured to or integral with the self-closing member 21, and more preferably to or with the second self-healing membrane 23, situated under the first self-healing membrane 22. The traction tab 27 thus advantageously forms an extension of the main body 30.

The invention also relates to a kit for deploying an intra-gastric balloon 1 that is designed to be implanted in the stomach of a patient by way of treatment for obesity.

In accordance with the invention, the kit comprises an intra-gastric balloon 1 of, the invention, and a connection member 11 for connecting the intra-gastric balloon 1 to an inflation fluid source, which connection member, in its storage configuration, is mounted in a manner such as to pass through the superposed self-healing membranes 22, 23 so as to open out inside the bag.

The connection member 11 advantageously includes an inflation needle 15 that is preferably formed by a hollow needle suitable for transpiercing the self-closing member 21 and the self-healing membranes 22, 23 in a manner such as to open out into the bag 2.

The design of the Intra-gastric balloon 1 of the invention thus enables the intra-gastric balloon 1 to be filled safely once it is disposed inside the stomach of the patient, with no risk of leakage, even after a prolonged period of storage of the intra-gastric balloon 1 with the inflation needle 15 pre-installed thereon.

Another advantage of the intra-gastric balloon 1 of the invention results from its ease of extraction procured by the presence of the cavity 31.

### SUSCEPTIBILITY OF INDUSTRIAL APPLICATION

The invention is susceptible of industrial application in manufacturing and using intra-gastric balloons for treating obesity.

1. An intra-gastric balloon designed to be implanted inside the stomach of a patient for the purpose of reducing the volume of the stomach by way of treatment for obesity, said intra-gastric balloon (1) comprising:
at least one bag (2) suitable for being filled, at least partially, with an inflation fluid; and
connection means (3), mounted on said bag (2) and including a self-closing member (21) suitable for being transpierced by a connection member (11) for connection to a inflation fluid source;
said intra-gastric balloon being characterized in that the self-closing member (21) comprises at least two superposed self-healing membranes (22, 23), forming one or more pairs of self-healing membranes (22, 23) suitable for being transpierced in succession by the connection member (11), each self-healing membrane (22, 23) being mounted in a manner such as to come to bear against the other self-healing membrane (22, 23) of the pair with a view to forming a leaktight barrier suitable for automatically closing the passage (24) formed through said other self-healing membrane (22, 23) of the pair, once the connection member (11) has been removed.

2. An intra-gastric balloon according to claim 1, characterized in that the self-healing membranes (22, 23) are formed of materials that have shape memory and that have substantially the same flexibility and elasticity properties in a manner such as to guarantee mutual leaktightness for them.

3. An intra-gastric balloon according to claim 1, characterized in that the self-healing membranes (22, 23) are made of the same material, and preferably of silicone.

4. An intra-gastric balloon according to claim 1, characterized in that the self-healing membranes (22, 23) are made of materials of different hardnesses.

5. An intra-gastric balloon according to claim 1, characterized in that the self-healing membranes (22, 23) are mounted to be free, at least locally, relative to each other, so as to make it possible, at least locally, for them to move along each other.

6. An intra-gastric balloon according to claim 1, characterized in that the self-healing membranes (22, 23) are attached to each other.

7. An intra-gastric balloon according to claim 6, characterized in that the self-healing membranes (22, 23) come to bear against each other via at least two contact faces (22B, 23B), said contact faces (22B, 23B) being fastened together, e.g. by adhesive bonding or by heat-sealing.

8. An intra-gastric balloon according to claim 7, characterized in that the contact faces (22B, 23B) are fastened together over substantially their entire area.

9. An intra-gastric balloon according to claim 1, characterized in that the self-closing member (21) further comprises a compression member (25), of the ring type, disposed in a manner such as to surround at least one of the self-healing membranes (22, 23) in a manner such as to compress it.

10. An intra-gastric balloon according to claim 9, characterized in that the pair of self-healing membranes (22, 23) is formed by a first self-healing membrane (22), compressed by the compression member (25) and a second self-healing membrane (23) not compressed by the compression member (25).

11. An intra-gastric balloon according to claim 9, characterized in that the self-closing member (21) includes a pull member (26) suitable for being manipulated for axially pulling the self-closing member (21) and for enabling the compression member (25) to be mounted around at least one of the self-healing membranes (22, 23).

12. An intra-gastric balloon according to claim 11, characterized in that the pull member (26) is formed by a traction tab (27) fastened to or formed integrally with one of the self-healing membranes (22, 23).

13. An intra-gastric balloon according to claim 1, characterized in that the connection means (3) include a main body (30) projecting into the intra-gastric balloon (1), said main body (30) including a top portion (30S) that is hollow so as to form a cavity (31), and a bottom portion (30I), containing the self-closing member (21).

14. An intra-gastric balloon according to claim 13, characterized in that the top portion (30S) is hollow over a distance sufficient for the wall of the main body (30) defining the cavity (31) to fold over while the top portion (30S) of the main body (30) is being pinched with a tool for extracting the intra-gastric balloon (1), such as endoscopic forceps.

15. An intra-gastric balloon according to claim 1, characterized in that the connection means (3) are provided with a collar (5) extending radially at the surface of the intra-gastric balloon (1) so as to form a graspable zone (35) via which the intra-gastric balloon (1) can be taken hold of by the extractor tool.

16. An intra-gastric balloon according to claim 15, characterized in that the graspable zone (35) is associated with a reinforcement (41) so as to form a reinforced graspable zone (35) dedicated to extracting the intra-gastric balloon (1) from the stomach.

17. A kit for deploying an intra-gastric balloon (1) designed to be implanted in the stomach of a patient by way of treatment for obesity, said kit comprising:
an intra-gastric balloon (1) according to claim 1; and
a connection member (11) for connecting the intra-gastric balloon (1) to the inflation fluid source, which connection member is, in its storage configuration, mounted in a manner such as to pass through the superposed self-healing membranes (22, 23) so as to open out into the bag (2).

18. A kit according to claim 17, characterized in that the connection member (11) includes an inflation needle (15) suitable for transpiercing the self-healing membranes (22, 23).
